(19) United States
(12) Patent Application Publication
(10) Pub. No.: US 2011/0201110 A1
(43) Pub. Date: Aug. 18, 2011

(54) EFFICIENT METHOD FOR ESTABLISHING INDUCED PLURIPOTENT STEM CELLS

(75) Inventors: Kenichi Tezuka, Gifu-shi (JP); Toshiyuki Shibata, Gifu-shi (JP); Takahiro Kunisada, Gifu-shi (JP); Naritaka Tamaoki, Gifu-shi (JP); Tomoko Takeda, Gifu-shi (JP); Shinya Yamanaka, Kyoto-shi (JP); Kazutoshi Takahashi, Kyoto-shi (JP)

(73) Assignees: GIFU UNIVERSITY, Gifu-shi (JP); KYOTO UNIVERSITY, Kyoto-shi (JP)

(21) Appl. No.: 13/056,526

(22) PCT Filed: Oct. 2, 2008
(86) PCT No.: PCT/JP2008/068320

§ 371 Date: May 10, 2011

Related U.S. Application Data
(60) Provisional application No. 61/085,308, filed on Jul. 31, 2008.

(51) Int. Cl. C12N 5/0775

(52) U.S. Cl. 435/366

(57) ABSTRACT

The present invention provides a method of producing induced pluripotent stem (iPS) cells, comprising bringing a nuclear reprogramming substance into contact with dental pulp stem cells. By using dental pulp stem cells as a source of somatic cells, the efficiency of establishment of human iPS cells by transfer of 3 or 4 factors can be improved dramatically. Additionally, dental pulp stem cells are easily available because they can be isolated and prepared from extracted wisdom teeth and teeth extracted because of periodontal disease and the like, so that they can be used widely as a source of somatic cells for iPS cell banks.

### TECHNICAL FIELD

The present invention relates to a method of improving the efficiency of establishment of induced pluripotent stem (hereinafter also referred to as “iPS”) cells and a use of dental pulp stem cells therefor.

### BACKGROUND ART

In recent years, mouse and human iPS cells have been established one after another. Takahashi and Yamanaka (1) induced iPS cells by introducing Oct3/4, Sox2, Klf4 and c-Myc genes into fibroblasts derived from a reporter mouse wherein a neomycin resistant gene is knocked-in into Fbx15 locus and forcing the cells to express the genes. Okita et al. (2) succeeded in the establishment of iPS cells (Nanog iPS cells) that show almost the same gene expression and epigenetic modification as those in embryonic stem (ES) cells by producing a transgenic mouse wherein green fluorescent protein (GFP) and puromycin-resistant genes are integrated into the locus of Nanog, whose expression is limited in pluripotent cells rather than Fbx15 expression, forcing the fibroblasts derived from the mouse to express the above-mentioned 4 genes and selecting puromycin-resistant and GFP-positive cells. Similar results were confirmed by other groups (3,4). Thereafter, it has been revealed that iPS cells can also be produced by 3 factors other than c-Myc gene (5).

Furthermore, Takahashi et al. (6) succeeded in the establishment of iPS cells by introducing the same 4 genes as used in mouse into human skin-derived fibroblasts. On the other hand, Yu et al. (7) produced human iPS cells using Nanog and Lin28 in place of Klf4 and c-Myc. Park et al. (8) produced human iPS cells using TERT and SV40 large T antigen known as immortalizing genes for human cells, in addition to 4 factors of Oct3/4, Sox2, Klf4 and c-Myc. As mentioned above, it has been demonstrated that iPS cells comparable to ES cells in pluripotency can be produced in both human and mouse.

However, the establishment efficiency of iPS cells is as low as 1%. Especially, a problem of extremely low establishment efficiency of iPS cells occurs when they are produced by introducing 3 factors (Oct3/4, Sox2 and Klf4) other than c-Myc, which is feared to cause tumorigenesis in tissues or individuals differentiated from the iPS cells, into somatic cells.

Regarding the establishment of human iPS cells by transfer of 3 or 4 factors, it has been reported to date that human iPS cells were established from adult human dermal fibroblasts or synovial cells, and from fetal or neonatal fibroblasts (see references 5, 6, 7, and 8). However, the efficiencies of their establishment are extremely low; for example, according to a study of Nakagawa et al. in which human iPS cells were established by transfer of 3 factors (Oct3/4, Sox2, Klf4) only (5), as few as 0 to 5 ES-cell-like colonies were obtained from 5×10⁵ adult human dermal fibroblasts (HDF).

Some reports are available on an improvement in the efficiency of establishment of iPS cells by transfer of 3 or 4 factors in mouse embryonic fibroblasts (MEF) using certain kinds of chemical substances (see, for example, references 9 and 10). However, there is no report that the efficiency of establishment of human iPS cells was remarkably improved merely by introducing 3 or 4 factors, without using such an establishment efficiency improver or any other nuclear reprogramming factor.

### REFERENCES

1. Takahashi, K. and Yamanaka, S., Cell, 126: 663-676 (2006)
2. Okita, K. et al., Nature, 448: 313-317 (2007)
3. Wernig, M. et al., Nature, 448: 318-324 (2007)
4. Maherali, N. et al., Cell Stem Cell, 1: 55-70 (2007)
5. Nakagawa, M. et al., Nat. Biotethnol., 26: 101-106 (2008)
6. Takahashi, K. et al., Cell, 131: 861-872 (2007)
7. Yu, J. et al., Science, 318: 1917-1920 (2007)
8. Park, I. H. et al., Nature, 451: 141-146 (2008)
9. Huangfu D. et al., Nat. Biotechnol., 26(7): 795-797 (2008)
10. Shi Y. et al., Cell Stem Cell, 2: 525-528 (2008)

### SUMMARY OF THE INVENTION

It is an object of the present invention to provide a means of improving the efficiency of establishment of iPS cells, and a method of efficiently producing iPS cells using the same.

It is another object of the present invention to provide a means of establishing iPS cells from relatively easily available cells.

The present inventors conducted extensive investigations with the aim of accomplishing the above-described objects, and found that the efficiency of establishment of iPS cells is remarkably increased by using dental pulp stem cells as the starting material somatic cells for preparation of human iPS cells (cells serving as a source of iPS cells). Specifically, the present inventors attempted to establish human iPS cells by introducing 3 factors (Oct3/4, Klf4, Sox2) or 4 factors (Oct3/4, Klf4, Sox2, c-Myc) into human dental pulp stem cells, and found for the first time that a much larger number of iPS cells can be established than that obtained conventionally from adult human dermal fibroblasts (HDF).

Accordingly, the present invention provides:

[1] A method of producing iPS cells, comprising bringing nuclear reprogramming substances into contact with dental pulp stem cells.
[2] The method according to [1] above, wherein the nuclear reprogramming substances are Oct3/4, Klf4 and Sox2, or nucleic acids that encode the same.
[3] The method according to [1] above, wherein the nuclear reprogramming substances are Oct3/4, Klf4, Sox2 and c-Myc, or nucleic acids that encode the same.
[4] The method according to [1] above, wherein the dental pulp stem cells are of human derivation.
[5] Use of dental pulp stem cells as a source of somatic cells for producing iPS cells.

Use of dental pulp stem cells makes it possible to remarkably increase the efficiency of establishment of iPS cells, and is therefore useful in inducing human iPS cells, for which the efficiency of establishment has conventionally been low, particularly in inducing iPS cells by transfer of 3 factors except c-Myc. Because c-Myc is feared to cause tumorigenesis when reactivated, the improvement in the efficiency of establishment of iPS cells using the 3 factors is of paramount utility in applying iPS cells to regenerative medicine.

Additionally, dental pulp stem cells are easily available because they can be isolated and prepared from extracted wisdom teeth, teeth extracted because of periodontal disease and the like, so that they are expected to find a new application for use as a source of somatic cells for iPS cell banks.

### BRIEF DESCRIPTION OF THE DRAWINGS

FIG. 1 shows graphs of the number of colonies of ES-like cells (iPS cells) obtained by reprogramming dental pulp stem cells. In FIG. 1, DP28, DP31, DP47, DP54, DP75, and DP87 show the results for dental pulp stem cells, and HDF shows the results for adult human dermal fibroblasts. Each axis of ordinates indicates the number of colonies. Each left bar shows the number of ES-like colonies; each right bar shows the total number of colonies. “3 factors at d26” shows the results obtained on day 26 after transfer of 3 factors (Oct3/4, Sox2, Klf4). “4 factors at d21” shows the results obtained on day 21 after transfer of 4 factors (Oct3/4, Sox2, Klf4, c-Myc).

FIG. 2 shows photographs demonstrating the results of an examination of gene expression in iPS cells derived from dental pulp stem cells. The expression of ES cell specific markers (Oct3/4, Sox2, Nanog) in iPS cells (iPS-DP31, iPS-DP75) derived from dental pulp stem cells (DP31, DP75) was confirmed by RT-PCR. In FIG. 2, 3f indicates clones prepared by transfer of 3 factors, and 4f indicates clones prepared by transfer of 4 factors. Each numerical figure under 3f and 4f indicates a clone number. “ES” indicates ES cells, “DP31” and “DP75” indicate dental pulp stem cells, “201B6” indicates iPS cells derived from adult human dermal fibroblasts (Cell, 131, p 861-872 (2007)), and “AHDF” indicates adult human dermal fibroblasts. NAT1 indicates a positive control, and RT-(OCT3/4) indicates a negative control (a PCR reaction of Oct3/4 was performed without performing a reverse transcription reaction).

FIG. 3, like FIG. 1, shows graphs of the number of colonies of ES-like cells (iPS cells). FIG. 3A shows the results of transfer of 3 factors (3F); FIG. 3B shows the results of transfer of 4 factors (4F). “4 ES like” and “4 total” show the number of ES-like colonies and the total number of colonies, respectively, obtained with 5×10⁴ dental pulp stem cells; “5 ES like” and “5 total” show the number of ES-like colonies and the total number of colonies, respectively, obtained with 5×10⁵ dental pulp stem cells.

FIG. 4 shows photographs demonstrating that an ES-like colony (iPS-DP47) established from dental pulp stem cells DP47 expresses the ES cell markers Nanog and Oct3/4 (Oct), as detected by immuno staining, and that the same is also positive for alkaline phosphatase (ALP) staining. For control, human ES cells (hES) were stained.

FIG. 5 shows photographs demonstrating the expression of stem cell markers (SSEA1, SSEA3, TRA-1-81 and NANOG) in two iPS clones (DP31 4f-3 and DP31 3f-1) established from dental pulp stem cells DP31.

FIG. 6 shows pluripotency of iPS cells derived from human dental pulp stem cells. FIG. 6A shows photographs demonstrating the formation of embryoid bodies in two iPS clones (DP31 4f-3 and DP31 3f-1) established from dental pulp stem cells DP31. FIG. 6B shows photographs demonstrating the expression of ectoderm- (βIII-tublin), mesoderm- (α-SMA) and endoderm- (AFP) differentiation markers in the iPS clones. control: secondary antibody only.

FIG. 7 shows photographs demonstrating the formation of teratomas derived from iPS cells established from human dental pulp stem cells. The teratomas were comprised of plural cell types such as adipose tissue (b), nerve tissue (c), intestinal tract-like tissue (d), cartilage tissue (e) and neural tube-like tissue (f). (a): overview of a teratoma.

### DETAILED DESCRIPTION OF THE INVENTION

The present invention provides a method of producing iPS cells, comprising bringing nuclear reprogramming substances into contact with dental pulp stem cells.

### (1) Dental Pulp Stem Cells

Being the source of somatic cells used in the method of producing iPS cells of the present invention, dental pulp stem cells are a kind of somatic stem cells which is present in dental pulp tissue inside the dentine of teeth, and which is capable of differentiating into dental pulp, dentine and the like (capable of differentiating mainly into odontoblasts). Dental pulp stem cells can be obtained by extirpating dental pulp tissue from (i) a tooth extracted for the sake of convenience in orthodontic treatment or a tooth extracted because of periodontal disease and the like, or from (ii) a wisdom tooth extracted for the sake of convenience in orthodontic treatment or of treatment of wisdom tooth periodontitis and the like, shredding the tissue into pieces of appropriate size, thereafter treating the pieces with an enzyme such as collagenase, sowing the resulting cell suspension to a culture medium for mesenchymal stem cells (see, for example, JP-T-HEI-11-506610 and JP-T-2000-515023; for example, mesenchymal stem cell basal medium (Lonza), MesenPRO RS Medium (GIBCO) and the like are commercially available), and culturing the cells by a conventional method.

Although any tooth retaining dental pulp tissue can be used as a source of dental pulp stem cells, it is preferable to select a tooth that is rich in dental pulp stem cells with a high potential for proliferation. In particular, when nuclear reprogramming substances are introduced to dental pulp stem cells using retroviral vectors, cells that permit retroviral transduction are limited to dividing cells. Therefore, it is desirable, from the viewpoint of gene transfer efficiency, that dental pulp tissue containing dental pulp stem cells with a high potential for proliferation be used as the starting material.

The most suitable source of dental pulp stem cells is dental pulp tissue derived from a wisdom tooth of a young person (for example, in humans, about 12-16 years) having the wisdom tooth extracted for orthodontic purposes. Wisdom teeth at these ages are still in the midst of dental root formation during the initial stage of dental differentiation, and are characterized by high abundance of dental pulp tissue, a relatively high density of dental pulp stem cells, and a very high potential for their proliferation.

Because wisdom teeth are sometimes extracted for orthodontic purposes in other age groups, and also because dental pulp tissue can be obtained also from teeth, other than wisdom teeth, extracted for the sake of convenience, the source availability is high.

Other potential sources of dental pulp stem cells include teeth extracted for the treatment of periodontal disease, wisdom teeth extracted because of wisdom tooth periodontitis and the like. In this case, there are disadvantages of an increased risk of contamination and a smaller amount of dental pulp tissue obtained. Because of the ease of obtainment from adults (particularly elderly), however, these materials can serve as a major source of dental pulp stem cells when autologous transplantation of cells or tissue differentiated from the iPS cells produced is taken into account.

It should be noted, however, that whenever an extracted tooth having a dental caries is chosen, its dental pulp tissue must not be affected by inflammation.

The dental pulp stem cells that can be used in the present invention may be derived from any animal species, including mammal, that permits the establishment of iPS cells by bringing nuclear reprogramming substances into contact with the dental pulp stem cells. Specifically, human or mouse dental pulp stem cells can be used, with preference given to those of human origin. Although dental pulp stem cells can be collected from any animal species, it is particularly preferable that the dental pulp stem cells be collected from the patient or from another person sharing the same type of HLA because of the absence of graft rejection, when the iPS cells obtained are used for human regenerative medicine. When the iPS cells are not administered (transplanted) to a human, but are used as, for example, a source of cells for screening to determine the presence or absence of the patient's drug susceptibility and adverse drug reactions, the dental pulp stem cells must be collected from the patient or from another person sharing the same gene polymorphism correlating to the drug susceptibility and adverse drug reactions.

Dental pulp stem cells prepared from an extracted tooth or a tooth that has dropped spontaneously, as described above, may be immediately brought into contact with nuclear reprogramming substances to induce iPS cells, or may be stored under freezing by a conventional method, thawed and cultured whenever necessary, and then brought into contact with nuclear reprogramming substances to induce iPS cells. Therefore, for example, it is also possible to prepare dental pulp stem cells from the patient's own deciduous tooth or permanent tooth or wisdom tooth extracted at a relatively young age, preserve them under freezing for a long time, induce iPS cells from therefrom when cell/organ transplantation is required later, and autologously transplant cells, tissue, organs and the like obtained by inducing the differentiation of the IFS cells.

### (2) Nuclear Reprogramming Substance

As used in the present invention, “nuclear reprogramming substance(s)” may be any substance(s) capable of inducing iPS cells from dental pulp stem cells, whether it is a proteinous factor or a nucleic acid that encodes the same (vector-incorporated forms included), a low-molecular compound or the like. When the nuclear reprogramming substances are proteinous factors or nucleic acids that encode the same, the following combinations are preferred (hereinafter, only the names of proteinous factors are shown).

(1) Oct3/4, Klf4, c-Myc
(2) Oct3/4, Klf4, c-Myc, Sox2 (here, Sox2 can be replaced with Sox1, Sox3, Sox15, Sox17 or Sox18. Klf4 can be replaced with Klf1, Klf2 or Klf5. Furthermore, c-Myc can be replaced with T58A (active mutant), N-Myc, or L-Myc.)
(3) Oct3/4, Klf4, c-Myc, Sox2, Fbx15, Nanog, Eras, ECAT15-2, TclI, β-catenin (active mutant S33Y)
(4) Oct3/4, Klf4, c-Myc, Sox2, TERT, SV40 Large T
(5) Oct3/4, Klf4, c-Myc, Sox2, TERT, HPV16 E6
(6) Oct3/4, Klf4, c-Myc, Sox2, TERT, HPV16 E7
(7) Oct3/4, Klf4, c-Myc, Sox2, TERT, HPV6 E6, HPV16 E7
(8) Oct3/4, Klf4, c-Myc, Sox2, TERT, Bmil
(See WO 2007/069666 (with respect to the combination (2) above, see Nature Biotechnology, 26, 101-106 (2008) for replacement of Sox2 with Sox18, replacement of Klf4 with Klf1 or Klf5); with respect to the combination “Oct3/4, Klf4, c-Myc, Sox2”, see also Cell, 126, 663-676 (2006), Cell, 131, 861-872 (2007) and the like; for the combination “Oct3/4, Klf4, c-Myc, Sox2, hTERT, SV40 Large T”, see also Nature, 451, 141-146 (2008)).

### (9) Oct3/4, Klf4, Sox2 (see Nature Biotechnology, 26, 101-106 (2008))

### (10) Oct3/4, Sox2, Nanog, Lin28 (see Science, 318, 1917-1920 (2007))

(11) Oct3/4, Sox2, Nanog, Lin28, hTERT, SV40 Large T (see Stem Cells Express, published online May 29, 2008, p 1-16)
(12) Oct3/4, Klf4, c-Myc, Sox2, Nanog, Lin28 (see Cell Research (2008) 600-603)
(13) Oct3/4, Klf4, c-Myc, Sox2, SV40 Large T (see also Stem Cells Express, published online May 29, 2008, p 1-16)
(14) Oct3/4, Klf4 (see Nature, Published online, 29 Jun. 2008, p 1-5 (doi:10.1038/nature07061))
(15) Oct3/4, c-Myc (see Nature, Published online, 29 Jun. 2008, p 1-5 (doi:10.1038/nature07061))

### (16) Oct3/4, Sox2 (see Nature, 451, 141-146 (2008))

Combinations other than (1)-(16) above, but comprising all constituents of any one thereof, and further comprising any other optionally chosen substance, can also be included in the scope of “nuclear reprogramming substances” in the present invention. Provided that the dental pulp stem cells express one or more constituents of any one of (1)-(16) above endogenously at a level sufficient to nuclear reprogramming, the combination of the remaining constituents only, except the constituents expressed, can also be included in “nuclear reprogramming substances” in the present invention.

Of these combinations, the combination of the 3 factors Oct3/4, Sox2 and Klf4 (i.e., (9) above) is preferable when use of the iPS cells obtained for therapeutic purposes is taken into account. Meanwhile, when use of the iPS cells for therapeutic purposes is not taken into account (for example, being used as an investigational tool for drug discovery screening and the like), preference is given to the 5 factors Oct3/4, Klf4, c-Myc, Sox2 and Lin28, or the 6 factors consisting of the five and Nanog (i.e., (12) above).

Mouse and human cDNA sequence information on the above-described individual proteinous factors can be acquired by reference to the NCBI accession numbers described in WO 2007/069666 (Nanog is therein mentioned under the designation “ECAT4”; mouse and human cDNA sequence information on Lin28 can be acquired with reference to the NCBI accession numbers NM_{—}145833 and NM_{—}024674, respectively), and those skilled in the art are easily able to isolate these cDNAs. When intended for use as a nuclear reprogramming substance, a proteinous factor per se can be prepared by inserting the cDNA obtained into an appropriate expression vector, introducing the vector to host cells, culturing the cells, and recovering the recombinant proteinous factor from the resulting culture. Meanwhile, when a nucleic acid that encodes a proteinous factor is used as a nuclear reprogramming substance, the cDNA obtained is inserted into a viral vector or a plasmid vector to construct an expression vector, and the vector is subjected to the step of nuclear reprogramming.

Contact of a nuclear reprogramming substance with dental pulp stem cells can be achieved using a method of protein transfer to cells known per se, provided that the substance is a proteinous factor. Such methods include, for example, the method using a protein transfer reagent, the method using a protein transfer domain (PTD) fusion protein, the microinjection method and the like. Protein transfer reagents are commercially available, including BioPOTER Protein Delivery Reagent (Gene Therapy Systems), Pro-Ject™ Protein Transfection Reagent (PIERCE) and ProVectin (IMGENEX), which are based on a cationic lipid; Profect-1 (Targeting Systems), which is based on a lipid; Penetrain Peptide (Q biogene) and Chariot Kit (Active Motif), which are based on a membrane-permeable peptide, and the like. The transfer can be achieved per the protocols attached to these reagents, the common procedures being as described below. A nuclear reprogramming substance is diluted in an appropriate solvent (for example, a buffer solution such as PBS or HEPES), a transfer reagent is added, the mixture is incubated at room temperature for about 5-15 minutes to form a complex, this complex is added to the cells after exchange with a serum-free medium, and the cells are incubated at 37° C. for one to several hours. Thereafter, the medium is removed and replaced with a serum-containing medium.

Developed PTDs include those using the cell penetrating domain of a protein such as drosophila-derived AntP, HIV-derived TAT, and HSV-derived VP22. A fusion protein expression vector incorporating a cDNA of the nuclear reprogramming substance and PTD sequence is prepared to allow recombinant expression of the fusion protein, and the fusion protein is recovered and used for the transfer. This transfer can be achieved as described above, except that no protein transfer reagents are added.

Microinjection, a method of placing a protein solution in a glass needle having a tip diameter of about 1 μm, and injecting the solution into a cell, ensures the transfer of the protein into the cell.

Taking into account the ease of transfer to dental pulp stem cells, it is preferable that the nuclear reprogramming substance be used in the form of a nucleic acid that encodes a proteinous factor, rather than of the proteinous factor per se. The nucleic acid may be a DNA or an RNA, or may be a DNA/RNA chimera, and the nucleic acid may be double-stranded or single-stranded. Preferably, the nucleic acid is a double-stranded DNA, particularly cDNA.

A cDNA of the nuclear reprogramming substance is inserted into an appropriate expression vector harboring a promoter capable of functioning in the dental pulp stem cells serving as the host. Useful expression vectors include, for example, viral vectors such as retrovirus, lentivirus, adenovirus, adeno-associated virus, and herpesvirus, plasmids for the expression in animal cells (e.g., pA1-11, pXT1, pRc/CMV, pRc/RSV, pcDNAI/Neo) and the like. The kind of vector used can be chosen as appropriate according to the intended use of the iPS cells obtained.

Useful promoters used in the expression vector include, for example, SRα promoter, SV40 promoter, LTR promoter, CMV (cytomegalovirus) promoter, RSV (Rous sarcoma virus) promoter, MoMuLV (Moloney mouse leukemia virus) LTR, HSV-TK (herpes simplex virus thymidine kinase) promoter and the like. Preference is given to MoMuLV LTR, CMV promoter, SRα promoter and the like.

The expression vector may harbor, as desired, in addition to a promoter, an enhancer, a polyadenylation signal, a selectable marker gene, an SV40 replication origin and the like. Examples of the selectable marker gene include the dihydrofolate reductase gene, the neomycin resistance gene and the like.

An expression vector harboring a nucleic acid being a nuclear reprogramming substance can be introduced to a cell by a technique known per se according to the kind of the vector. In the case of a viral vector, for example, a plasmid containing the nucleic acid is introduced to appropriate packaging cells (e.g., Plat-E cells) or a complementary cell line (e.g., 293-cells), the viral vector produced in the culture supernatant is recovered, and the vector is infected to the cell by a method suitable for the viral vector. In the case of a plasmid vector, the vector can be introduced to a cell using the lipofection method, liposome method, electroporation method, calcium phosphate co-precipitation method, DEAE dextran method, microinjection method, gene gun method and the like.

When the nuclear reprogramming substance is a low-molecular compound, contact of the substance with dental pulp stem cells can be achieved by dissolving the substance at an appropriate concentration in an aqueous or non-aqueous solvent, adding the solution of the substance to a medium suitable for cultivation of dental pulp stem cells (for example, a minimal essential medium (MEM), Dulbecco's modified Eagle medium (DMEM), RPMI1640 medium, 199 medium, F12 medium and the like supplemented with about 5 to 20% fetal calf serum; or a medium for mesenchymal stem cells such as mesenchymal stem cell basal medium (Lonza)) to obtain a concentration of the nuclear reprogramming substance that is sufficient to cause nuclear reprogramming in the dental pulp stem cells, but is not cytotoxic, and culturing the cells for a given period. The concentration of the nuclear reprogramming substance varies depending on the kind of the nuclear reprogramming substance used, and is chosen as appropriate in the range of about 0.1 nM to about 100 nM. Length of the contact may be any time sufficient to achieve nuclear reprogramming of the cells.

### (3) iPS Cell Establishment Efficiency Improver

In recent years, various substances that improve the efficiency of establishment of iPS cells have been proposed, which efficiency has conventionally been low. It can be expected, therefore, that the efficiency of establishment of iPS cells will be further increased by bringing, in addition to the above-described nuclear reprogramming substances, these establishment efficiency improvers into contact with dental pulp stem cells.

Examples of iPS cell establishment efficiency improvers include, but are not limited to, histone deacetylase (HDAC) inhibitors [for example, valproic acid (VPA) (Nat. Biotechnol., 26(7): 795-797 (2008)), low-molecular inhibitors such as trichostatin A, sodium butyrate, MC 1293, and M344, nucleic acid-based expression inhibitors such as siRNA and shRNA against HDAC (e.g., HDAC1 siRNA Smartpool® (Millipore), HuSH 29mer shRNA Constructs against HDAC1 (OriGene) and the like), and the like], G9a histone methyltransferase inhibitors [for example, low-molecular inhibitors such as BIX-01294 (Cell Stem Cell, 2: 525-528 (2008)), nucleic acid-based expression inhibitors such as siRNA and shRNA against G9a (e.g., G9a siRNA (human) (Santa Cruz Biotechnology) and the like) and the like] and the like. The nucleic acid-based expression inhibitors may be in the form of expression vectors harboring a DNA that encodes siRNA or shRNA.

Of the aforementioned constituents of nuclear reprogramming substances, SV40 large T, for example, can also be included in the scope of iPS cell establishment efficiency improvers because they are auxiliary factors unessential for the nuclear reprogramming of somatic cells. While the mechanism of nuclear reprogramming remains unclear, it does not matter whether auxiliary factors, other than the factors essential for nuclear reprogramming, are deemed nuclear reprogramming substances, or deemed iPS cell establishment efficiency improvers. Hence, because the somatic cell nuclear reprogramming process is visualized as an overall event resulting from contact of nuclear reprogramming substances and an iPS cell establishment efficiency improver with somatic cells, it does not always seem necessary for those skilled in the art to distinguish both.

Contact of an iPS cell establishment efficiency improver with dental pulp stem cells can be achieved in the same manner as the method described above with respect to nuclear reprogramming substances, when the improver is (a) a proteinous factor, (b) a nucleic acid that encodes the proteinous factor, or (c) a low-molecular compound, respectively.

The iPS cell establishment efficiency improver may be brought into contact with dental pulp stem cells simultaneously with the nuclear reprogramming substances, or either may be brought into contact in advance, as far as the efficiency of establishment of iPS cells from dental pulp stem cells is significantly improved compared to the level obtained in the absence of the improver. In an embodiment of the present invention, when the nuclear reprogramming substances are nucleic acids that encode proteinous factors, and the iPS cell establishment efficiency improver is a chemical inhibitor, for example, the former involves a given length of time lag between gene transfer treatment and mass expression of the proteinous factors, whereas the latter is capable of quickly acting on cells, so that the iPS cell establishment efficiency improver can be added to the medium after the cells are cultured for a given time following the gene transfer treatment. In another embodiment of the present invention, when the nuclear reprogramming substances and the iPS cell establishment efficiency improver are both used in the form of a viral vector or plasmid vector, for example, both may be introduced to the cells simultaneously.

Dental pulp stem cells can be pre-cultured using a medium known per se which is suitable for the cultivation thereof (see, for example, JP-T-HEI-11-506610, JP-T-2000-515023; for example, mesenchymal stem cell basal medium (Lonza), MesenPRO RS Medium (GIBCO) and the like are commercially available). The dental pulp stem cells can also be pre-cultured using, for example, a minimal essential medium (MEM), Dulbecco's modified Eagle medium (DMEM), RPMI1640 medium, 199 medium or F12 medium and the like supplemented with about 5 to 20% fetal calf serum.

When a transfer reagent such as cationic liposome, for example, is used in achieving contact with nuclear reprogramming substances (and an iPS cell establishment efficiency improver), it is sometimes preferable to previously replace the medium with a serum-free medium to prevent the transfer efficiency from decreasing. After contact with a nuclear reprogramming substances (and an iPS cell establishment efficiency improver), the cells can be cultured under, for example, conditions suitable for the cultivation of ES cells. In the case of human cells, it is preferable that the cells be cultured in the presence of basic fibroblast growth factor (bFGF) added as a differentiation suppressor to an ordinary medium. In the case of mouse cells, it is desirable that leukemia inhibitory factor (LIF) be added in spite of bFGF. Usually, the cells are cultured in the presence of mouse embryonic fibroblasts (MEF), previously treated with radiation or antibiotics to terminate cell division, as feeder cells. Although STO cells and the like are commonly used as the MEF, SNL cells (McMahon, A. P. & Bradley, A. Cell 62, 1073-1085 (1990)) and the like are commonly used for induction of iPS cells.

There are two approaches of selecting a candidate colony of iPS cells: use of drug resistance and/or reporter activity as indicator(s) and macroscopic examination of morphology. In the former approach, for example, a colony that is positive for drug resistance and/or reporter activity is selected using recombinant dental pulp stem cells wherein a drug resistance gene and/or a reporter gene has been targeted to the gene locus of a gene that is highly expressed specifically in pluripotent cells (for example, Fbx15, Nanog, Oct3/4 and the like, preferably Nanog or Oct3/4). Meanwhile, methods of macroscopic examination of morphology include, for example, the method described by Takahashi et al. in Cell, 131, 861-872 (2007). Although methods using reporter cells are convenient and efficient, colony selection by macroscopic examination is desirable from the viewpoint of safety when iPS cells are prepared for the purpose of human treatment. When the 3 factors Oct3/4, Klf4 and Sox2 are used as nuclear reprogramming substances, the number of clones established decreases, but the resulting colonies are for the most part iPS cells whose quality is as high as that of ES cells; therefore, it is possible to efficiently establish iPS cells even without using reporter cells. In particular, the present invention is effective in dramatically improving the efficiency of establishment of iPS cells by transfer of 3 factors, thus making it possible to select a candidate colony of iPS cells at sufficient efficiency by macroscopic examination of morphology.

The identity of the cells of the selected colony as iPS cells can be confirmed by a variety of testing methods known per se, for example, the ES-cell-specific gene expression analysis described in an Example below and the like. If greater accuracy is wanted, the selected cells may be transplanted to a mouse and examined for teratoma formation.

The iPS cells thus established can be used for a broad range of purposes. For example, by means of a method of differentiation induction reported for ES cells, it is possible to induce the differentiation of iPS cells into a wide variety of types of cells (e.g., myocardial cells, retinal cells, blood cells, nerve cells, vascular endothelial cells, insulin secreting cells and the like), tissues and organs.

Because dental pulp stem cells can be prepared from teeth/wisdom teeth extracted by corrective surgery, teeth/wisdom teeth extracted because of dental caries, periodontal disease, wisdom tooth periodontitis and the like, and the like, it is possible to easily collect dental pulp stem cells from a large number of persons (a dental pulp stem cell bank is currently available). Therefore, the dental pulp stem cells of the present invention can be used extremely effectively as a source for preparing (1) individual persons' iPS cells or (2) iPS cells corresponding to multiple HLA antigen types.

If transplantation of cells or tissue to a patient is urgently demanded, it is sometimes no use preparing iPS cells from the patient's somatic cells, and allowing them to differentiate, after onset of the disease. In preparation for such cases, the above-described problem can be solved to enable transplantation even in emergency by (1) previously preparing a bank of iPS cells from individual persons' somatic cells or cells or tissue differentiated therefrom, or by (2) previously preparing a bank of iPS cells, or cells or tissue differentiated therefrom, for each HLA antigen type. The dental pulp stem cells of the present invention can also be used effectively in tailor-made regenerative medicine or semi-tailor-made regenerative medicine like this.

Furthermore, because functional cells (e.g., hepatocytes) differentiated from iPS cells are thought to better reflect the actual state of the functional cells in vivo than do corresponding existing cell lines, they can also be suitably used for in vitro screening for the effectiveness and toxicity of pharmaceutical candidate compounds and the like.

The present invention is hereinafter described in greater detail by means of the following examples, to which, however, the invention is never limited.

### EXAMPLES

### Example 1

### Establishment of iPS Cells from Human Dental Pulp Stem Cells (1)

### Experimental Procedures

Dental pulp stem cells were prepared from teeth extracted from persons at 12 to 24 years of age (DP28, DP31, DP47, DP54, DP75, DP87). Specifically, pulp tissue was extirpated from each wisdom tooth extracted from an orthodontic patient or a patient with wisdom tooth periodontitis, and shredded using ophthalmologic Cooper scissors into about 1 to 2 mm tissue pieces, after which the tissue pieces were treated with collagenase type I (1 mg/ml) at 37° C. for 0.5 to 1 hour. This was cultured in a mesenchymal stem cell basal medium (produced by Lonza) to establish a cell line of dental pulp stem cell. For control, adult human dermal fibroblasts (HDF) from a 36-year-old person were also prepared. These cells were allowed to express the mouse ecotrophic virus receptor Slc7a1 gene using lentivirus as directed in Cell, 131, 861-872 (2007).

Four (Oct3/4, Sox2, Klf4, c-Myc) or three (Oct3/4, Sox2, Klf4) human-derived factors were introduced to these cells (8×10⁵ cells) by means of retrovirus by the method described in Cell, 131, 861-872 (2007). Six days after the viral infection, the cells were recovered, and re-sown onto feeder cells (5×10⁴ or 5×10⁵ cells/100 mm dish). The feeder cells used were SNL cells (McMahon, A. P. & Bradley, A. Cell 62, 1073-1085 (1990)), previously treated with mitomycin C to terminate their cell division. On the following day, the cells were transferred to a medium for primate ES cell culture (ReproCELL), supplemented with 4 ng/ml recombinant human bFGF (WAKO), and cultured.

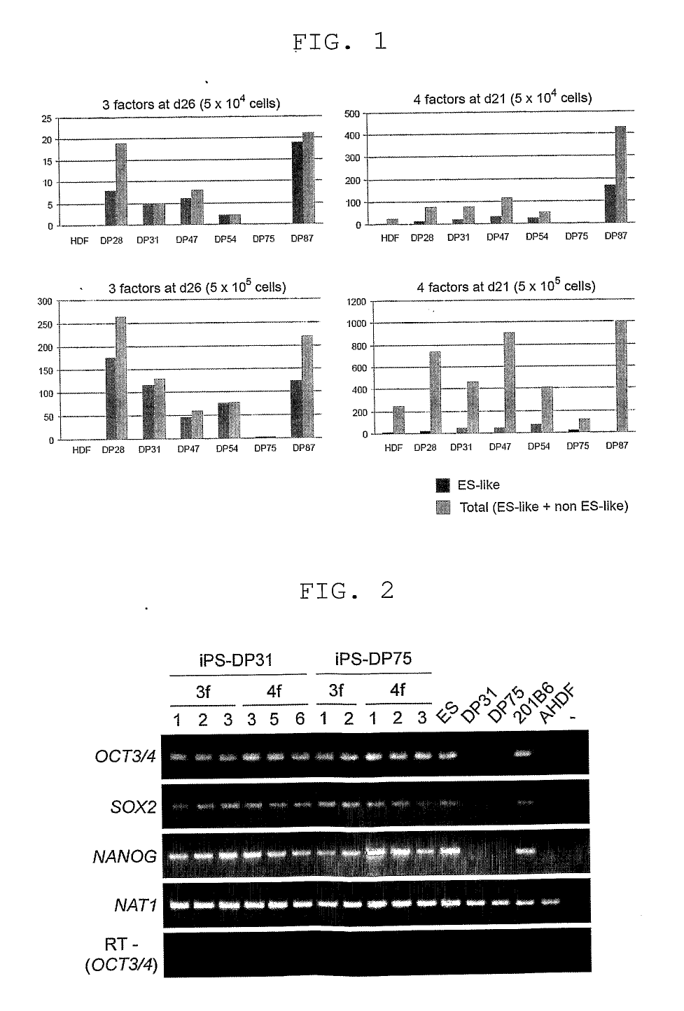

For the cells incorporating the 4 factors, colonies that emerged on day 21 after the retroviral infection were counted. The colonies were morphologically evaluated and counted in two types: ES-like cells (iPS cells) and non ES-like cells (non-iPS cells). The results are shown in Table 1 and FIG. 1 (FIG. 1 is a graphic representation of Table 1).

TABLE 1

| | | | day 26 | | day 21 | |
|---|---|---|---|---|---|---|
| | | | 3 factors | | 4 factors | |
| | age (M/F) | cell count | es like | total | es like | total |
| HDF | 36F | 5 × 10\hat{ }5 | 0 | 0 | 10 | 246 |
| | | 5 × 10\hat{ }4 | 0 | 0 | 2 | 24 |
| DP28 | 14M | 5 × 10\hat{ }5 | 176 | 263 | 19 | 734 |
| | | 5 × 10\hat{ }4 | 8 | 19 | 11 | 74 |
| DP31 | 14F | 5 × 10\hat{ }5 | 116 | 129 | 42 | 465 |
| | | 5 × 10\hat{ }4 | 5 | 5 | 20 | 75 |
| DP47 | 12F | 5 × 10\hat{ }5 | 46 | 59 | 42 | 903 |
| | | 5 × 10\hat{ }4 | 6 | 8 | 30 | 116 |
| DP54 | 19M | 5 × 10\hat{ }5 | 76 | 77 | 76 | 410 |
| | | 5 × 10\hat{ }4 | 2 | 2 | 24 | 50 |
| DP75 | 24M | 5 × 10\hat{ }5 | 1 | 1 | 18 | 117 |
| | | 5 × 10\hat{ }4 | 0 | 0 | 0 | 4 |
| DP87 | 20F | 5 × 10\hat{ }5 | 123 | 218 | 0 | >1000 |
| | | 5 × 10\hat{ }4 | 19 | 21 | 169 | 428 |

In five of the 6 lines of dental pulp stem cells incorporating the 4 factors, ES-like colonies were obtained at 2 to 8 times higher efficiencies when the cell count was 5×10⁵ cells, and at 5 to 80 times higher efficiencies when the cell count was 5×10⁴ cells, compared to dermal fibroblasts (Table 1, right panels in FIG. 1).

For the cells incorporating the 3 factors, colonies that emerged on day 26 after the retroviral infection were counted. For the dermal fibroblasts incorporating the 3 factors, no colonies were observed on day 26, whereas for 5 of the 6 lines of dental pulp stem cells, 2 to 19 ES-like colonies (from 5×10⁴ cells) or 46 to 176 ES-like colonies (from 5×10⁵ cells) were obtained (Table 1, left panels in FIG. 1).

The iPS cells established from dental pulp stem cells, like those established from dermal cells, exhibited a morphology resembling that of human ES cells, and were capable of proliferating continuously on the feeder cells.

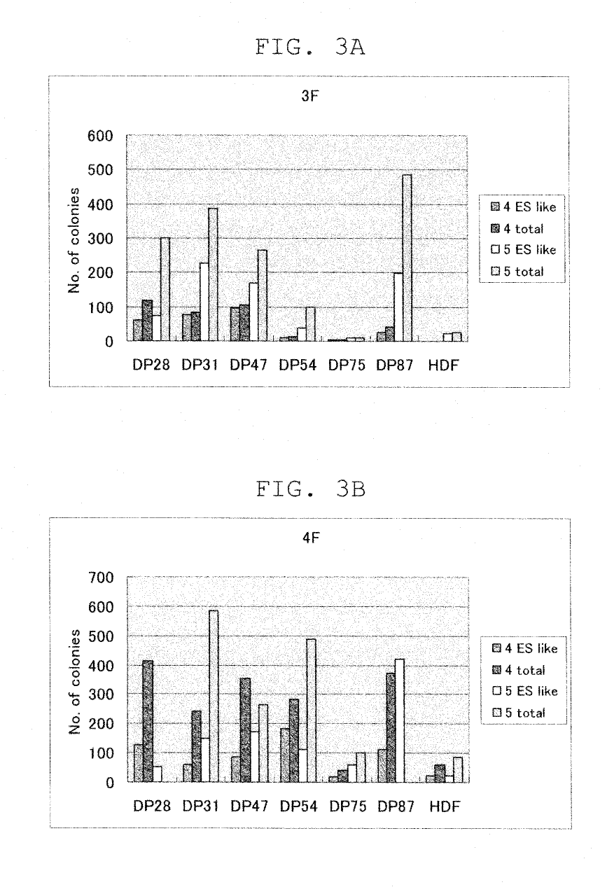

An RT-PCR analysis using the Rever Tra Ace kit (Takara) showed that the ES-like colonies established from DP31 and DP75 expressed the human ES cell specific marker genes Oct3/4, Sox2, and Nanog, and that the amounts of expression thereof were equivalent to those obtained with human ES cells and dermal iPS cells established in the past (201B6) (FIG. 2). These results identified the cells established from dental pulp stem cells as iPS cells.

### Example 2

### Establishment of iPS Cells from Human Dental Pulp Stem Cells (2)

### Experimental Procedures

iPS cells were established by introducing 4 or 3 factors to the same dental pulp stem cells in the same manner as those in Example 1.

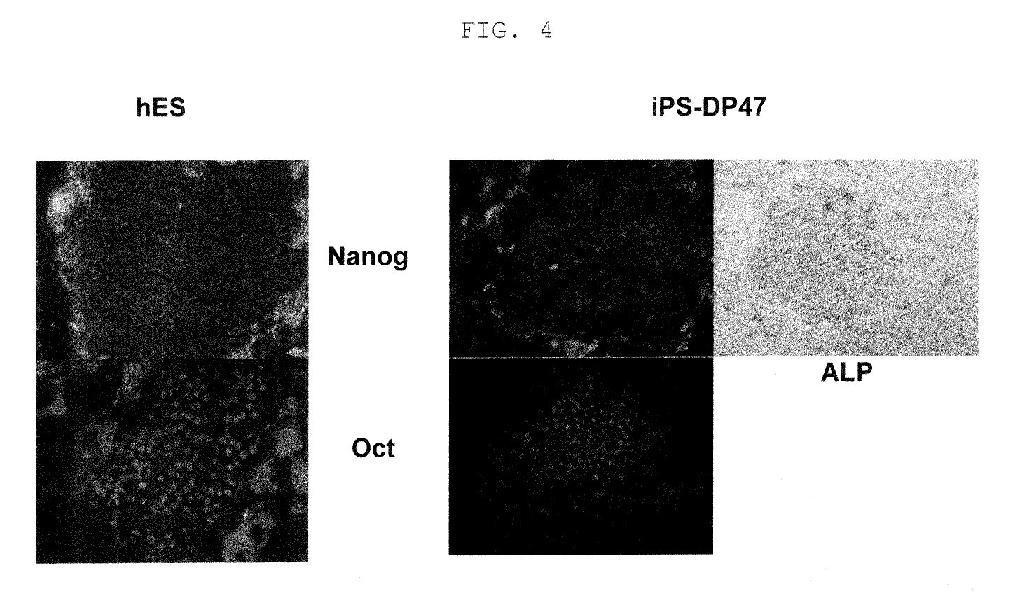

For the cells incorporating the 4 factors, the colonies that had emerged were counted when ES-like colonies could be picked up for each line after retroviral infection. The colonies were morphologically evaluated and counted in two types: ES-like cells (iPS cells) and non ES-like cells (non-iPS cells). The results are shown in FIG. 3.

In five of the 6 lines of dental pulp stem cells incorporating the 4 factors, ES-like colonies were obtained at 2 to 19 times higher efficiencies when the cell count was 5×10⁵ cells, and at 3 to 9 times higher efficiencies when the cell count was 5×10⁴ cells, compared to HDF (FIG. 3B).

For the cells incorporating the 3 factors, in 5 of the 6 lines of dental pulp stem cells, ES-like colonies were obtained at 2 to 10 times higher efficiencies when the cell count was 5×10⁵ cells, compared to HDF, and no ES-like colonies emerged with HDF when the cell count was 5×10⁴ cells. By contrast, in all the 6 lines of dental pulp stem cells, ES-like colonies were produced, with as many as nearly 200 colonies emerging in some lines (FIG. 3A).

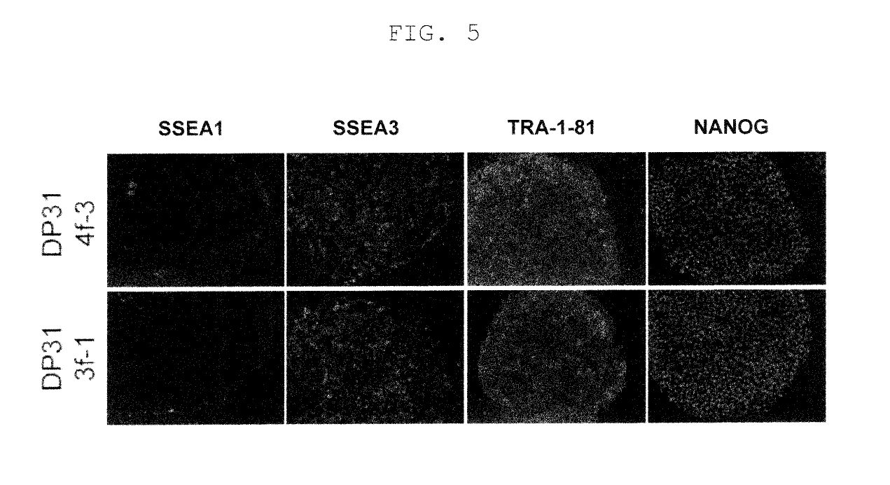

The ES-like colonies established from DP47 were examined for the expression of the ES cell markers Nanog and Oct3/4 by immunological staining. The antibodies used were anti-Nanog produced by R&D Systems, and anti-Oct3/4 produced by Santa Cruz Biotechnology. As a result, the expression of both factors was confirmed (FIG. 4). The colonies established tested positive for alkaline phosphatase activity (FIG. 4). These results identified the cells established from the dental pulp stem to cells as iPS cells.

### Example 3

### Stem Cell Marker Expression in iPS cells

### Experimental Procedures

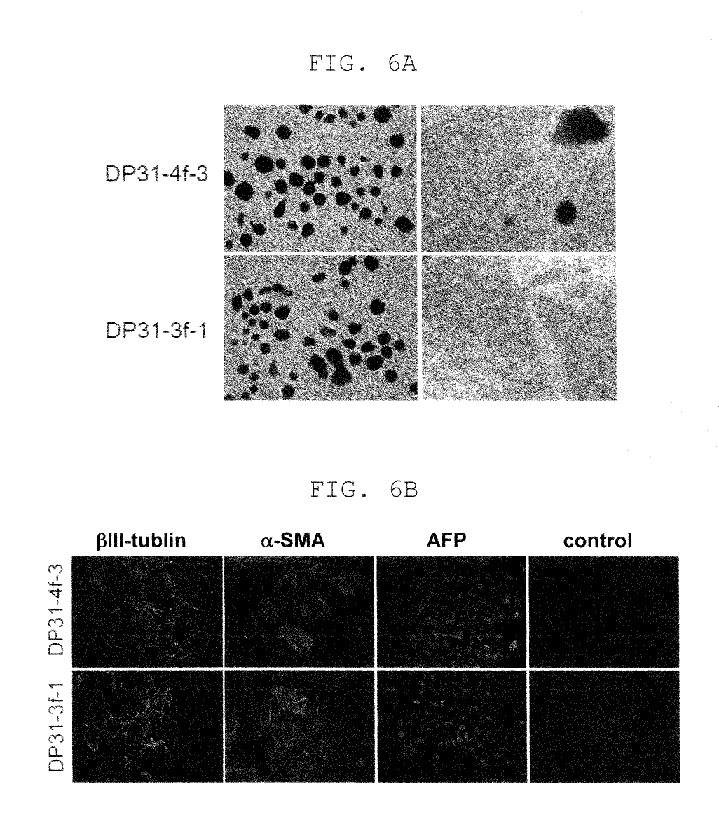

The iPS cells obtained in Example 1 were plated onto mitomycin C-treated SNL feeder cells and incubated for 5 days. The cells were fixed with 4% paraformaldehyde and permeabilized and blocked with PBS containing 5% normal goat serum, 1% BSA and 0.2% TritonX-100. The expression of stem cell markers (SSEA1, SSEA3, TRA-1-81, NANOG) was examined by immunocytochemistry. As primary antibodies, anti-SSEA1 (1:100, Developmental Studies Hybridoma Bank of Iowa University), anti-SSEA3 (1:100, a gift from Dr. Peter Andrews), TRA-1-81 (1:100, a gift from Dr. Peter Andrews) and anti-NANOG (1:20, R&D systems) were used. Secondary antibodies used were as follows; Alexa 488-labeled anti-mouse IgM (1:500, Invitrogen), Cy3-labeled anti-rat IgM (1:500, Jackson Immunoresearch) and Alexa-546-labeled anti-goat IgG (1:500, Invitrogen). Nuclei were stained with Hoechst 33342 (Invitrogen). The results are shown in FIG. 5. All of iPS clones analyzed expressed SSEA3, TRA-1-81 and NANOG proteins. In contrast, most of the cells were not stained with anti-SSEA1 antibody, though the positive cells were observed at the edge of some colonies. Similar expression patterns of human ES cells and iPS cells were previously reported. These data suggested that iPS cells derived from human dental pulp stem cells were also comparable to ES cells in undifferentiated ES cell marker expression.

### Example 4

### Pluripotency of iPS Cells Derived from Human Dental Pulp Stem Cells

### Experimental Procedures

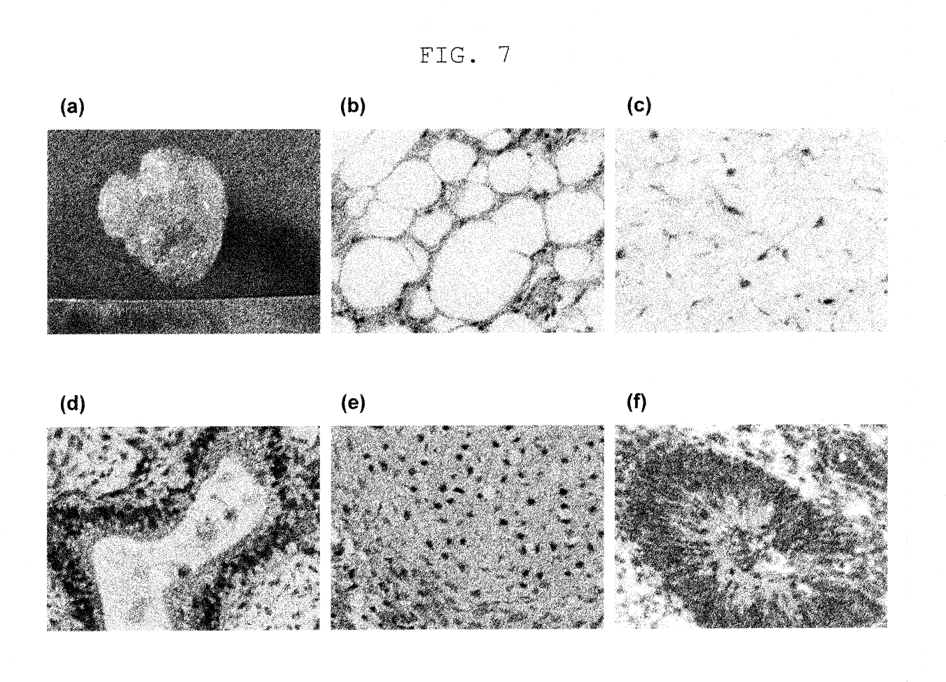

Next, the present inventors confirmed whether these iPS cells were pluripotent by in vitro differentiation. To form embryoid bodies, the cells were harvested and transferred to poly-hydroxyethyl methacrylate (HEMA)-coated dishes and incubated for 8 days. After floating culture, the embryoid bodies formed were plated onto gelatin-coated plates and incubated for another 8 days. After incubation, the cells were fixed with 4% paraformaldehyde and permeabilized and blocked with PBS containing 5% normal goat serum, 1% BSA and 0.2% TritonX-100. The expression of differentiation markers (βIII-tublin, α-SMA, AFP) was examined by immunocytochemistry. As primary antibodies, anti-(βIII-tublin (1:100, Chemicon), anti-α-smooth muscle actin (α-SMA) (1:500, DAKO) and anti-α-fetoprotein (AFP) (1:100, R&D systems) were used. Cy3-labeled anti-mouse IgG (1:500, Chemicon) was used as secondary antibody. Nuclei were stained with Hoechst 33342 (Invitrogen). The results are shown in FIG. 6. Eight days after floating culture, the iPS cells formed embryoid bodies (FIG. 6A). After incubation on the gelatin-coated plates, the cells changed morphologically to various cell types. Immunocytochemistry showed that the iPS cells differentiated into three germ layers such as ectoderm (βIII-tublin), mesoderm (α-SMA) and endoderm (AFP) (FIG. 6B). No significant difference in differentiation potentials was found between the iPS clones.

### Example 5

### Teratoma Formation

### Experimental Procedures

The present inventors further analyzed pluripotency of iPS cells by teratoma formation assays. The cells were treated with 10 μM Y-27632 (Wako) for 1 hour, and then harvested. The cells were suspended at approximately 1×10′ cells/ml in DMEM/F12 supplemented with 10 μM Y-27632. Thirty microliters of the cell suspension was injected into testes of Severe Combined Immunodeficiency (SCID) mouse (Charles River) by using Hamilton syringe. Two or 3 months after injection, teratomas were dissected and fixed with PBS containing 10% formalin. Paraffin-embedded samples were sliced and stained with hematoxylin and eosin. The results are shown in FIG. 7. The teratomas were comprised of plural cell types including adipose tissue, nerve tissue, intestinal tract-like tissue, cartilage tissue and neural tube-like tissue, which demonstrated pluripotency of the iPS cells.

While the present invention has been described with emphasis on preferred embodiments, it is obvious to those skilled in the art that the preferred embodiments can be modified. The present invention intends that the present invention can be embodied by methods other than those described in detail in the present specification. Accordingly, the present invention encompasses all modifications encompassed in the gist and scope of the appended “CLAIMS.”

The contents disclosed in any publication cited here, including patents and patent applications, are hereby incorporated in their entireties by reference, to the extent that they have been disclosed herein.

1. A method of producing induced pluripotent stem cells, comprising bringing nuclear reprogramming substances into contact with dental pulp stem cells.

2. The method of claim 1, wherein the nuclear reprogramming substances are Oct3/4, Klf4 and Sox2, or nucleic acids that encode the same.

3. The method of claim 1, wherein the nuclear reprogramming substances are Oct3/4, Klf4, Sox2 and c-Myc, or nucleic acids that encode the same.

4. The method of claim 1, wherein the dental pulp stem cells are of human derivation.

5. Use of dental pulp stem cells as a source of somatic cells for producing induced pluripotent stem cells.
